(19) United States
(12) Patent Application Publication
(10) Pub. No.: US 2015/0374942 A1
(43) Pub. Date: Dec. 31, 2015

(54) TRACHEOSTOMY TUBE ASSEMBLIES AND INNER CANNULAE

(71) Applicant: SMITHS MEDICAL INTERNATIONAL LIMITED, Ashford, Kent (GB)

(72) Inventors: Timothy Bateman, Kent (GB); Stephen James Field, Kent (GB); Christopher Stratton Turnbull, Kent (GB)

(73) Assignee: SMITHS MEDICAL INTERNATIONAL LIMITED, Kent (GB)

(21) Appl. No.: 14/767,637

(22) PCT Filed: Feb. 5, 2014
(86) PCT No.: PCT/GB2014/000042

§ 371 Date: Aug. 13, 2015

(30) Foreign Application Priority Data
Feb. 28, 2013 (GB) 1303554.8

(51) Int. Cl. A61M 16/04

(52) U.S. Cl. CPC A61M 16/0488

(57) ABSTRACT

A tracheostomy tube assembly includes an outer tracheal tube (1) and a removable inner cannula (20). The inner cannula (20) has a corrugated outer surface (23) but a smooth inner surface (22). At its patient end the inner cannula (20) has an integral tip (26) with a smooth outer surface that forms a close sliding fit with the inside of the outer tube (1). At its rear, machine end the inner cannula has an integral hub (30) that fits in a hub (18) on the outer tube. The smooth inner surface (22) of the inner cannula (20) allows non-turbulent gas flow along the assembly and facilitates insertion of devices along the cannula.

This invention relates to inner cannulae of the kind for replaceable insertion in a tracheostomy tube.

Tubes are used in many medical and non-medical applications. For example, tracheostomy tube assemblies commonly include an outer tube and an inner tube or cannula that is a removable fit within the outer tube. The inner cannula can be removed and replaced periodically to ensure that the passage through the assembly does not become blocked by secretions. This avoids the need to remove the outer tube frequently.

The inner cannula presents various problems because it must be thin walled and a close fit within the outer tube so as to provide a large bore and thereby limit the resistance to flow of gas along the assembly. It must, however, also be sufficiently stiff to be inserted in the outer tube without buckling or kinking. WO94/01156 and WO2004/101048 describe inner cannulae made of PTFE. EP1938857 describes an arrangement of tracheostomy tubes and inner cannulae where the hubs of the inner cannulae of different sizes are shaped differently so that they will only fit in the appropriate tracheostomy tube. EP2224985 describes an arrangement for attaching a hub to the shaft of an inner cannula. GB2056285 describes an inner cannula having a wall corrugated both externally and internally and a longitudinal groove or other reinforcement member traversing at least some of the corrugations. U.S. Pat. No. 4,817,598 describes a smooth-walled inner cannula having a ring-pull formation at its rear, machine end. U.S. Pat. No. 5,119,811 describes an inner cannula with a flared patient end and formed of two layers of different materials. U.S. Pat. No. 5,386,826 describes an inner cannula with an outer helical filament or layer of low friction material. U.S. Pat. No. 5,983,895 describes an inner cannula with straight sections at opposite ends joined by an intermediate curved section. U.S. Pat. No. 6,019,753 describes an inner cannula with two elongate regions of different flexibility so that the cannula has a plane of preferential bending. U.S. Pat. No. 6,019,753 describes an inner cannula having a shaft formed with slots to make it more flexible, the slots being covered by an outer thin sheath. U.S. Pat. No. 6,135,110 describes a curved inner cannula that is retained with the outer tube by means of a rotatable spring fitting.

It is an object of the present invention to provide an alternative inner cannula and tracheostomy tube assembly.

According to one aspect of the present invention there is provided an inner cannula of the above-specified kind, characterised in that the inner cannula is formed with a substantially smooth inner surface and an outer surface having generally circumferential corrugations.

The corrugations may be of annular form or helical form. The inner cannula may include a reinforcement element extending longitudinally of the cannula across a plurality of corrugations such as to increase the axial strength of the cannula against compression. The inner cannula is preferably moulded with an integral hub at its machine end. The inner cannula is preferably moulded with an integral tip portion at its patient end, the tip portion having a smooth external surface.

The inner cannula preferably includes a reinforcement element extending longitudinally across a plurality of corrugations such as to increase the axial strength of the cannula against compression. The cannula is preferably moulded with an integral hub at its rear, machine end. The cannula is preferably moulded with an integral tip portion at its forward, patient end having a smooth external surface.

According to another aspect of the present invention there is provided a tracheostomy tube assembly including an outer tracheostomy tube and an inner cannula according to the above one aspect of the present invention, the inner cannula being inserted within the outer tube and being removable from the outer tube.

The inner cannula is preferably formed with a hub at its machine end and a tip portion with a smooth external surface at its patient end, the hub on the inner cannula fitting in a hub on the outer tube, and the tip portion of the inner cannula being a close sliding fit with the inside of the outer tube.

A tracheostomy tube assembly including an inner cannula in accordance with the present invention, will now be described, by way of example, with reference to the accompanying drawings, which are not to scale, and in which:

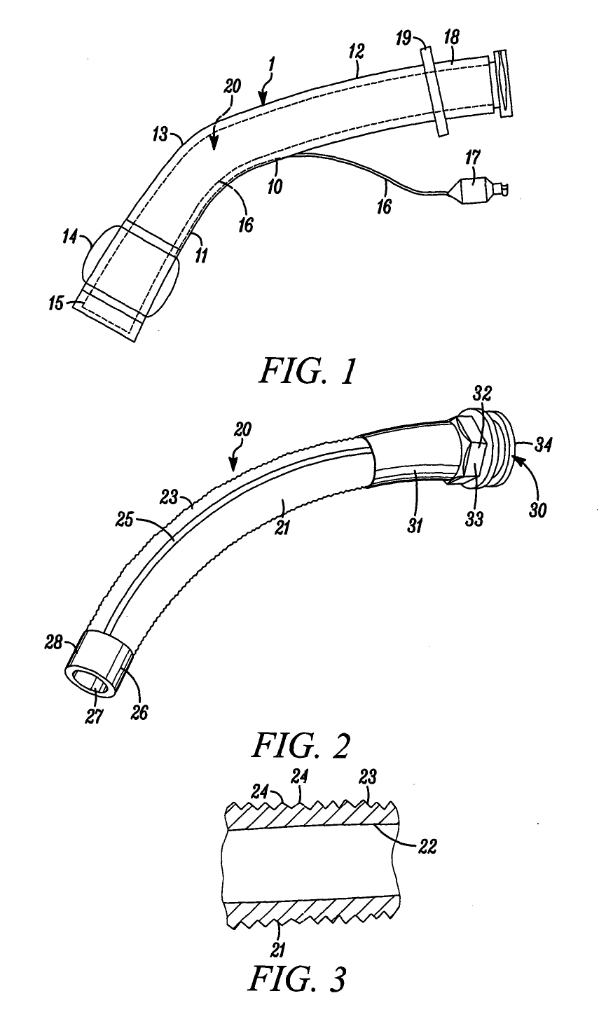

FIG. 1 is a side elevation view of a tracheostomy tube assembly including an inner cannula;

FIG. 2 is a perspective view of the inner cannula; and

FIG. 3 is a cross-sectional view through the inner cannula along the line of FIG. 2.

With reference first to FIG. 1, the tracheostomy tube assembly comprises an outer tracheostomy tube 1 and a removable inner cannula 20 inserted within the outer tube. The outer tube 1 has a shaft 10 with a straight forward section 11, a straight rear section 12 and a curved intermediate section 13 linking the forward and rear sections. An inflatable sealing cuff 14 embraces the forward section 11 close to the patient end 15 of the tube, the cuff being inflated via an inflation lumen 16 and a combined connector and inflation indicator 17. At its rear end the outer tube 1 has a hub 18 and flange 19 to which a retaining tape can be fastened for securing the tube with the patient's neck. The inside of the hub 18 is formed with keying flats (not shown), of the kind described in EP1938857, adapted to prevent full insertion of an inner cannula of the wrong size. The outer tube 1 could have an internal diameter between about 6 mm and 10 mm, and its length could be between 60 mm and 200 mm.

With reference now also to FIGS. 2 and 3, the inner cannula 20 is moulded from a plastics material such as by blow moulding or injection moulding. The main part of the length of the cannula 20 is provided by a shaft 21 of circular section and having a smooth inner surface 22 and an outer surface 23 that is circumferentially corrugated with corrugations 24. The corrugations 24 typically have a V-shape profile but could have alternative profiles, such as of sine wave shape. The corrugations could be annular, forming a complete circle, or could be formed by adjacent turns of a helical form (the term “circumferential” is intended to include a helical locus as well as annular formations). The outer diameter of the shaft 21 (that is, measured across the peaks of the corrugations) is selected to be the same as the internal diameter of the outer tube 1 so that the inner cannula 20 is a close sliding fit within the outer tube. The wall thickness of the shaft 21 is about 0.25 mm, which is thinner than conventional inner cannula shafts which are typically between about 0.5 mm and 1 mm. The shaft 21 is also formed with a longitudinal reinforcing element in the form of a spine 25 extending along its length along the outside of the curvature of the cannula. The spine 25 is preferably an integral formation in the form of a thickened strip traversing the corrugations moulded as a single piece with the shaft 21 itself but it could be formed by the addition of an extra component subsequently attached to the shaft, or insert moulded with the shaft. The reinforcing element need not be formed by the addition of material but could be provided by a groove formed by a flattened region traversing the corrugations.

The forward or patient end of the inner cannula 20 is moulded with an integral tip portion 26 as one piece with the shaft, the length of the tip portion being substantially equal to the outer diameter of the shaft 21. The tip portion 26 is smooth both on its inner surface 27 and on its external surface 28 so that it makes a close sliding fit with the inside of the outer tube 1. This tip portion 26 is stiffer than the remainder of the shaft 21 to help ensure that it maintains a circular shape and an effective seal with the inside of the outer tube 1 at its tip 15.

At its rear or machine end the inner cannula 20 is moulded with an integral hub portion 30 as one piece with the shaft 21. The hub portion 30 has a smooth external surface, the portion 31 at its forward end being shaped to fit into a 15 mm connector. To the rear of the forward portion 31 the hub has a keying portion 32 provided with flats 33 of the kind described in EP1938857 adapted to fit with corresponding formations in the hub 18 of the outer tube 1. At its rear end the inner cannula 20 has a ring-pull formation 34 of the kind described in U.S. Pat. No. 4,817,598, which facilitates removal of the inner cannula from the outer tube 1 after use.

The flexibility of the inner cannula 20 is such that it can readily bend to follow the shape of the different sections 12, 13 and 11 during insertion into the outer tube 1 without buckling or kinking.

The smooth inner surface of the shaft 21 allows for smooth, non-turbulent flow of gas along the assembly. It also allows devices, such as endoscopes, probes, suction catheters or the like to be inserted freely along the cannula without the risk of catching, as might be the case with a cannula having a corrugated inner surface. The corrugated external surface of the inner cannula 20 helps enhance the radial rigidity of the cannula and reduce the risk of kinking and buckling whilst still ensuring that the inner cannula can bend freely during insertion in and withdrawal from the outer tube 1. The corrugations 24 form a close fit within the outer tube 1 but the low area of contact ensures a low friction for insertion and removal. The corrugations 24 also help wipe any secretions that might collect in the outer tube 1 when the inner cannula 20 is withdrawn and replaced. Furthermore, the corrugations 24 help form a labyrinth seal with the inside surface of the outer tube 1 to reduce escape of gas between the outside of the inner cannula 20 and the inside of the outer tube.

Although the inner cannula 20 is described as being an integral, one-piece moulding with the tip portion 26 and the hub portion 30, it could, instead, be formed of separate components. For example, the corrugated shaft could be extruded and the hub and tip portions subsequently attached with the shaft by overmoulding.

1-8. (canceled)

9. An inner cannula for replaceable insertion in a tracheostomy tube, characterized in that the inner cannula is formed with a substantially smooth inner surface and an outer surface having generally circumferential corrugations.

10. An inner cannula according to claim 9, characterized in that the corrugations are of annular form.

11. An inner cannula according to claim 9, characterized in that the corrugations are of helical form.

12. An inner cannula according to claim 9, characterized in that the inner cannula includes a reinforcement element extending longitudinally of the cannula across a plurality of corrugations such as to increase the axial strength of the cannula against compression.

13. An inner cannula according to claim 9, characterized in that the inner cannula is molded with an integral hub at its machine end.

14. An inner cannula according to claim 9, characterized in that the inner cannula is molded with an integral tip portion at its patient end, and that the tip portion has a smooth external surface.

15. A tracheostomy tube assembly including an outer tracheostomy tube and an inner cannula formed with a substantially smooth inner surface and an outer surface having generally circumferential corrugations wherein the inner cannula is inserted within the outer tube and being removable from the outer tube.

16. A tracheostomy tube assembly according to claim 15, characterized in that the inner cannula is formed with a hub at its machine end and a tip portion with a smooth external surface at its patient end, that the hub on the inner cannula fits in a hub on the outer tube, and that the tip portion of the inner cannula is a close sliding fit with the inside of the outer tube.

17. An inner cannula according to claim 15, characterized in that the corrugations are of annular form.

18. An inner cannula according to claim 15, characterized in that the corrugations are of helical form.

19. An inner cannula according to claim 15, characterized in that the inner cannula includes a reinforcement element extending longitudinally of the cannula across a plurality of corrugations such as to increase the axial strength of the cannula against compression.

20. An inner cannula according to claim 15, characterized in that the inner cannula is molded with an integral hub at its machine end.

21. An inner cannula according to claim 15, characterized in that the inner cannula is molded with an integral tip portion at its patient end, and that the tip portion has a smooth external surface.
